(19) United States
(12) Patent Application Publication
(10) Pub. No.: US 2006/0257536 A1
(43) Pub. Date: Nov. 16, 2006

(54) Method for determining a moment in a preparation process of food at which the food has reached a ready state

(75) Inventor: Jan Tiesinga, Drachten (NL)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics N.V., Eindhoven (NL)

(21) Appl. No.: 10/568,018

(22) PCT Filed: Aug. 3, 2004
(86) PCT No.: PCT/IB04/51373

§ 371 Date: Feb. 10, 2006

(51) Int. Cl. G01N 33/02

(52) U.S. Cl. 426/231

(57) ABSTRACT

A method for determining a moment in a preparation process of food at which the food. has reached a ready state, the method comprising the following steps:—determining a initial water content, of the food;—determining a final water content of the food, which is associated with the ready state of the food;—determining an actual quantity of the food;—determining a. required quantity of released water, i.e. a quantity of water to be released during the preparation process in order for the food to reach the ready state, on the basis of the determinedd actual quantity of the food and a difference between the initial water content of the food and the final water content of the food; and—performing measurements during the preparation process in order to determine a moment on which an actual quantity of released water corresponds to the required quantity A preparation device (1) for subjecting food to a preparation process, comprising:—weighing means (20) for performing weighing processes and—controlling means for registering and processing results of the weighing processes as performed by the weighing means (20), wherein the controlling means are programmed such as to perform the above method.

The present invention relates to a method for determining a moment in a preparation process of food at which the food has reached a ready state. In the preceding as well as in the following, the term “ready state” pertains to a predetermined extent to which the food is done. It should be noted that when it is said that the food has reached the ready state, this does not necessarily mean that the food is fit for consumption. The preparation process during which the method according to the invention is carried out may be one step of a process during which food is made fit for consumption, wherein the one step does not necessarily need to constitute the last step of this process.

The method according to the present invention may for example be incorporated in a fryer. According to the state of the art, a person who applies a fryer to fry food must regularly check the state of the food which is inside the fryer, and must determine on sight or by taking out and tasting a sample of the food whether the food is ready or not, at one or more moments in the frying process. The conventional fryer may comprise a timer, which may be set by a user of the fryer on the basis of experience and/or an evaluation of the properties of the frying process, such as a quantity of food and a frying temperature.

The conventional method for checking whether the food has reached the ready state may be regarded as a trial-and-error method. A person applying the conventional method is only able to find a moment in the frying process when the food is ready by inspecting the appearance of the food and by tasting a sample of the food. In the process, it may even appear that the person starts the inspection too late, as a result of which the food may be too well done, have bad taste, be of a bad quality or be too dry. In the worst case, the food may even be burned. In a case in which a timer is applied in order to indicate a set preparation time, it may occur that the food is not ready when the timer generates a signal in order to indicate the end of the preparation time, and it may also occur that the food is dried out or even burned. The reason for this is that the person can misjudge the amount of food to be fried and/or the properties of the frying process, which properties include required preparation time and required temperature.

The conventional way of checking whether the food is ready has many disadvantages. It is bothersome to check the state of the food by checking the appearance and taste of the food, especially when the checking procedure needs to be repeated a few times during a frying process. Furthermore, as already mentioned in the foregoing, there is a danger of the food getting spoiled.

It is an objective of the present invention to provide a method for determining a moment in a preparation process of food at which the food has reached a ready state, wherein objective criteria are applied, and dependency on the judgment of a person is excluded. The objective is achieved by a method, comprising the following steps:

determining a initial water content of the food;

determining a final water content of the food, which is associated with the ready state of the food;

determining an actual quantity of the food;

determining a required quantity of released water, i.e. a quantity of water to be released during the preparation process in order for the food to reach the ready state, on the basis of the determined actual quantity of the food and a difference between the initial water content of the food and the final water content of the food; and

performing measurements during the preparation process in order to determine a moment on which an actual quantity of released water corresponds to the required quantity of released water.

The present invention is based on the insight that the ready state of food is related to the water content of the food. In fact, a preparation process such as frying or baking may be regarded as a process during which water is abstracted from the food. In the process, the food is heated, in order to release the water from the food by means of evaporation.

In fact, according to the present invention, a new quality parameter of prepared food is introduced, which is closely related to the water content of food, and which may be referred to as juiciness. The food has reached a predetermined ready state as soon as the food has obtained a predetermined juiciness.

According to the present invention, an initial water content of the food and a final water content of the food are determined, wherein the latter is associated with the ready state of the food. An actual quantity of the food is determined, so that it is possible to determine a required quantity of released water, i.e. a quantity of water to be released during the preparation process in order for the food to reach the ready state. In other words, it is possible to determine which quantity of water should be released by the food in order for the water content of the food to decrease to the level of the final water content which is associated with the ready state of the food. The moment when the food is ready is determined by performing measurements which are aimed at determining whether an actual quantity of released water corresponds to the required quantity of released water.

For the purpose of determining the actual quantity of released water, the released water may be collected. This is not necessary; it is for example also possible to determine the actual quantity of released water by measuring a weight loss of the food. In a preferred way of carrying out the method according to the invention, the initial water content and the final water content are used to determine the extent to which the weight of the food needs to be reduced in order for the food to reach the ready state. At the start of the preparation process, an initial weight of the food is measured. On the basis of information regarding the initial weight of the food and the required reduction of the weight of the food, it is possible to determine a required final weight of the food. The moment when the food is ready is determined by constantly measuring an actual weight of the food and comparing the actual weight of the food to the required final weight of the food. As soon as the actual weight of the food corresponds to the required final weight of the food, the water content of the food has reached the final level at which the food is regarded as ready.

The method according to the present invention is accurate and reliable. Directly or indirectly measuring the actual quantity of released water provides useful information regarding the state of the food. In order to determine whether the food has reached the ready state, it is not necessary to measure other parameters, like the temperature at which the food is prepared. In case the preparation process takes place in a preparation medium which contains no water, such as oil, it is safe to assume that the actual released water is only originating from the food. Well-known examples of such a preparation process are a baking process and a frying process.

Advantageously, the method according to the present invention comprises additional steps, which are aimed at ensuring that the food obtains a predetermined crispness and/or brown level. In particular, the crispness and/or brown level are related to the state of a jacket of the food. During a preparation process of food, the crispness and/or brown level are mainly determined by an applied temperature profile, i.e. a prescribed course which is followed by the temperature during the time of the preparation process. As soon as a relation between the temperature profile and the crispness and/or brown level of the food is defined, wherein the relation may be in the form of a collection of combinations of the temperature profile on the one hand and the crispness and/or brown level on the other hand, it is possible to determine a temperature profile which needs to be realized during the preparation process in order for the food to obtain a predetermined crispness and/or brown level. During the preparation process, the temperature is continuously controlled and adjusted, so that the temperature profile is actually realized, and the predetermined crispness and/or brown level are obtained.

It will be understood that the speed of evaporation of water from the food during the preparation process is related to the temperature. In general, it is true that the speed of evaporation increases when the temperature increases. Therefore, in a practical application of the present invention, the temperature profile is mainly determined on the basis of a desired crispness and/or brown level of the food, wherein the influence of the temperature profile on the speed of evaporation of water from the food is accounted for, in order to avoid as much as possible a situation in which the food reaches the ready state before or after the desired crispness and/or brown level are obtained. For example, the following steps may be performed:

determining a speed at which the water is released from the food;

estimating an actual duration of the preparation process on the basis of the determined speed and the difference between the initial water content of the food and the final water content of the food;

comparing the estimated actual duration of the preparation process to a duration of the preparation process that is required on the basis of the temperature profile; and, in case a difference between the estimated actual duration of the preparation process and the required duration of the preparation process is found,

adjusting the temperature profile such that it is possible for the food to substantially simultaneously reach the ready state and the predetermined crispness and/or brown level.

If automatic lifting means are present, the controller may be programmed to check the speed of the evaporation of water from the food during the frying process, and to check whether there is enough time to obtain the desired crispness and/or brown level. If it turns out that the speed of the evaporation of water is too high, it may be necessary to adjust the temperature profile in order to still obtain the desired crispness and/or brown level. In the process, the basket 14 may be lifted from the oil bowl 15 while the temperature of the oil is adjusted. After the oil temperature has been adjusted, the basket 14 may be lowered again.

In EP 1 183 974, a cooking apparatus is disclosed, which comprises a controller for storing a reference weight change corresponding to a water content change to attain a target state of food materials cooked in a cooking vessel of the cooking apparatus. During a cooking process of food materials, the controller controls a water content in the cooking vessel, or a ratio of the weight of water within and around food materials in the cooking vessel to the weight of all materials in the cooking vessel, so that it is possible to finish the food materials with a required taste such as sweetness or saltiness. The controller also monitors a water content in the cooking vessel so that food materials therein are not burned.

An important principle underlying the present invention, i.e. the insight that the ready state of food is related to the water content of the food, is not applied in the cooking apparatus as disclosed in EP 1 183 974. Instead, in the disclosed cooking apparatus, the water content is controlled in order to obtain a predetermined ratio of various ingredients of the food materials. Therefore, a required weight change which is stored in the controller of the cooking apparatus is merely associated to said predetermined ratio of the ingredients of the food materials, and is not associated to the ready state of food in the sense of the present invention.

The method according to the present invention is not applicable in cases of for example mixtures containing water, like soup or sauce, whereas the known cooking apparatus is specially intended for preparing such types of food. Consequently, in respect of the known cooking apparatus, workability is improved if the quantities of the components of the mixture are predetermined through tests. The present invention specifically pertains to preparing one type of food, wherein a small amount of additives like herbs may be added to this one type of food, and wherein the ready state is directly related to the water content of the food. The food to be subjected to the method according to the present invention may for example be a portion of potatoes, French fries, spring rolls, chicken legs, fish, etc.

In EP 1 183 974, specific measures for realizing a predetermined crispness and/or brown level of the food are not disclosed. As far as the temperature is concerned, it is disclosed that the temperature is set at a high level, in order to enable high-speed and high-temperature cooking.

The present invention will now be explained in greater detail with reference to the figures, in which similar parts are indicated by the same reference signs, and in which:

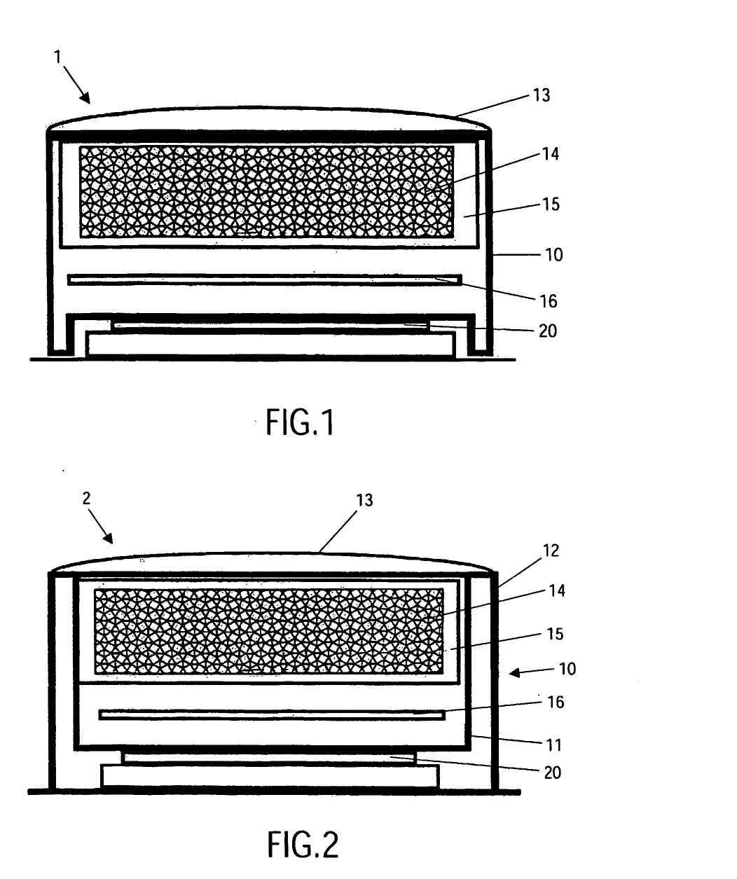

FIG. 1 diagrammatically shows a first preferred embodiment of a fryer in which the method according to the present invention is incorporated;

FIG. 2 diagrammatically shows a second preferred embodiment of a fryer in which the method according to the present invention is incorporated;

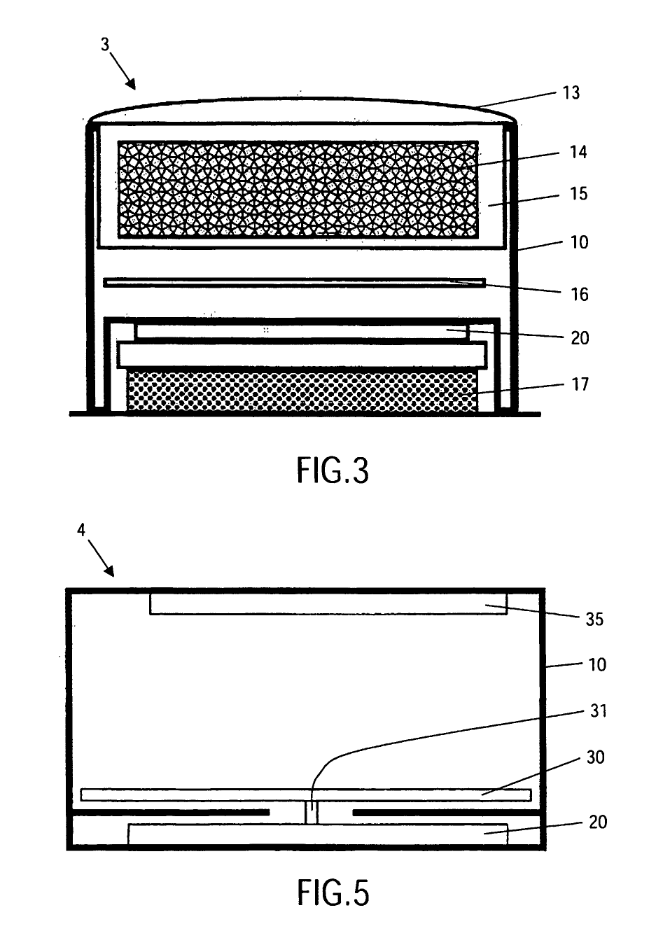

FIG. 3 diagrammatically shows a third preferred embodiment of a fryer in which the method according to the present invention is incorporated;

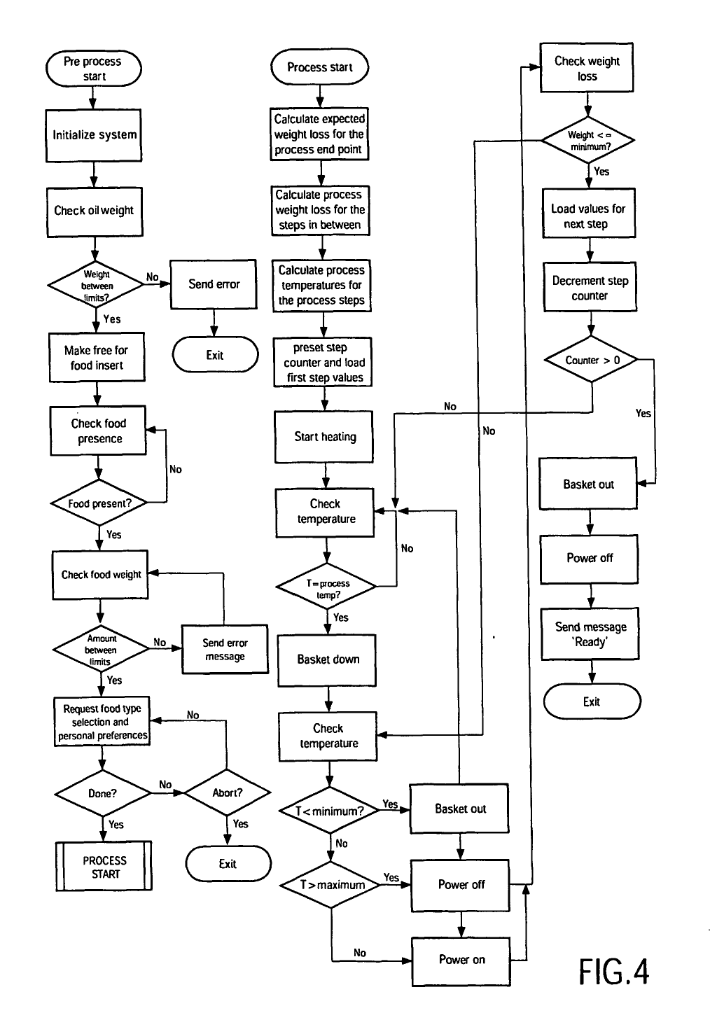

FIG. 4 is a flowchart of an algorithm which may be applied when carrying out the method according to the invention; and

FIG. 5 diagrammatically shows an embodiment of a microwave oven in which the method according to the present invention is incorporated.

In FIG. 1, a first fryer 1 for subjecting food to a frying process is shown, which comprises a housing 10 and a cover 13 for covering a top side of the housing 10. Preferably, one side of the cover 13 is pivotably connected to the housing 10, but the cover 13 may for example also be a separate component which can entirely be removed from the housing 10.

For the purpose of receiving and containing food, the first fryer 1 comprises a basket 14, which is preferably a separate component which can entirely be removed from the first fryer 1, so that food can easily be put into the basket 14 before the start of a frying process and taken out of the basket 14 after the end of the frying process.

Inside the housing 10, a space 15 is present, which is capable of receiving the basket 14, and which may be filled with oil or another suitable preparation medium. In the following, the space 15 in the housing 10 will be referred to as oil bowl 15.

For the purpose of heating the contents of the oil bowl 15, the first fryer 1 is provided with heating elements 16. In the shown example, the heating elements 16 are positioned underneath the oil bowl 15.

According to an important aspect of the present invention, the first fryer 1 comprises a scale 20, which is positioned underneath the housing 10. In this configuration, the scale 20 is capable of measuring the total weight of the other fryer components, the oil and the food, assuming that the oil bowl 15 is filled with oil and the basket 14 is filled with food.

The scale 20 is connected to controlling means (not shown), which register and process the values of the total weight of the other fryer components, the oil and the food. The controlling means are designed such as to use these values in a process of determining a moment in the frying process when the food is ready, which process will be explained later. The controlling means may for example comprise a microcontroller.

In FIG. 2, a second fryer 2 for subjecting food to a frying process is shown. An important difference between the second fryer 2 as shown in FIG. 2 and the first fryer 1 as shown in FIG. 1 relates to the housing 10. The housing 10 of the second fryer 2 comprises an inner housing 11 and an outer housing 12, wherein the inner housing 11 is located inside the outer housing 12. The oil bowl 15 and the heating elements 16 are located inside the inner housing 11, whereas the scale 20 is located outside the inner housing 11, but inside the outer housing 12. An important advantage of the second fryer 2 over the first fryer 1 is that the scale 20 is protected by the outer housing 12, as a result of which the risk of disturbance or damage of the scale 20 is substantially reduced.

In the configuration of the second fryer 2, the scale 20 is capable of measuring the total weight of the inner housing 11, the fryer components which are enclosed by the inner housing 11, the oil and the food, assuming that the oil bowl 15 is filled with oil and the basket 14 is filled with food.

In FIG. 3, a third fryer 3 for subjecting food to a flying process is shown. An important difference between the third fryer 3 as shown in FIG. 3 and the first fryer 1 as shown in FIG. 1 is that the third fryer 3 additionally comprises a condenser 17 for collecting the water (steam) which is released during the frying process. As a result of the presence of the condenser 17, during a frying process, steam does not leave the third fryer 3.

In the third fryer 3, the scale 20 is positioned above the condenser 17. Like in the first fryer 1, the scale 20 is capable of measuring the total weight of the housing 10, the cover 13, the basket 14, the oil bowl 15, the heating elements 16, the oil and the food, assuming that the oil bowl 15 is filled with oil, and the basket 14 is filled with food and placed in the oil bowl 15.

In an alternative embodiment, the housing 10 of the third fryer 3 may comprise an inner housing 11 and an outer housing 12, in the same manner as the housing 10 of the second fryer 2 comprises an inner housing 11 and an outer housing 12.

In the following, the way in which the first fryer 1 may be applied is described, and the way in which the controlling means of the first fryer 1 work is explained. By way of illustration, a flowchart of an algorithm which may be laid down in the controlling means of the first fryer 1 is shown in FIG. 4.

In the controlling means, the value of the total weight of the housing 10, the cover 13, the basket 14, the oil bowl 15 and the heating elements 16, in other words, the total weight of all fryer components above the scale 20, is stored. At the start of a frying process, the scale 20 is activated to perform a first measurement, wherein the scale 20 measures a total weight of the said components of the first fryer 1 and the oil in the oil bowl 15. The result of the first measurement is transmitted to the controlling means, which determine the value of the weight of the oil by calculating a difference between the stored value of the total weight of the fryer components above the scale 20 and the measured value.

As soon as the value of the weight of the oil in the oil bowl 15 is determined, the basket 14 may be taken out, filled with food and placed back in the oil bowl 15. When the filled basket 14 is in place, the controlling means activate detection means (not shown) in order to detect the presence of food. If it appears that food is present, the scale 20 is activated to perform a second measurement, wherein the scale 20 measures a total weight of the fryer components above the scale 20, the oil in the oil bowl 15 and the food in the basket 14. The result of the second measurement is transmitted to the controlling means, which determine the value of an initial weight of the food by calculating a difference between the weight value as measured during the first measurement and the weight value as measured during the second measurement.

In order for the controlling means to be able to control the frying process according to the desires of a user, the controlling means must be provided with information associated with these desires, as well as with information regarding the type of the food which is inserted. For this purpose, the first fryer 1 is provided with input means (not shown), which are to be set by a user of the first fryer 1. The input means may for example comprise three rotatable buttons, wherein the position of a first button is specific of the type of food which is inserted, wherein the position of a second button is specific of a desired ready state, and wherein the position of a third button is specific of a desired crispness and/or brown level of the food. The desired ready state may for example be indicated as medium, done, well done.

In the controlling means, information regarding an initial water content of the possible different types of food is stored. Furthermore, the controlling means contain information regarding a final water content of the possible different types of food, which final water content is associated with a ready state of the food. In order to be able to realize different ready states, different values of the final water content of the food are stored in the controlling means. In case of the frying process comprising at least two steps, a set of different values is associated with each of the steps. On the basis of the input which is given by the user, the controlling means are able to find an associated final water content. As soon as a user has indicated the type of food which is inserted, the controlling means are able to find the associated initial water content of the food and the final water content of the food. On the basis of the value of the initial weight of the food, which has already been determined by the controlling means, and a difference between the initial water content and the final water content of the food, the controlling means are able to determine a required final weight of the food. The required final weight of the food is defined as the weight of the food at which the water content of the food corresponds to the final water content, in other words, the weight of the food at which the food is considered to be ready.

During the frying process, the weight of the oil does not significantly change. Naturally, the same is true for the weight of the fryer components. Therefore, the controlling means are able to determine a reference value of the weight as detected by the scale 20, which is associated with the required final weight of the food. The reference value is simply found by adding the total weight of the fryer components above the scale 20 and the weight of the oil in the oil bowl 15 to the required final weight of the food.

An actual value of the weight as detected by the scale 20 is regularly checked and compared to the reference value throughout the frying process. As soon as it appears that the actual value of the weight corresponds to the reference value, or that the actual value of the weight is below the reference value, the controlling means stop the frying process. At that point, the water content of the food has reached the level of the final water content which is associated with the ready state of the food, which means that the food has actually reached the ready state.

When the second fryer 2 or the third fryer 3 is applied, the method for determining at which moment of the frying process the food is ready is performed in the same manner as is described in respect of the application of the first fryer 1. The scale 20 of the second fryer 2 weighs different fryer components than the scale 20 of the first fryer 1, but this fact does not influence the fundamental way in which the method according to the invention is carried out.

In the third fryer 3, the water (steam) which is released during the frying process is collected in the condenser 17. It is therefore also possible that a modified third fryer 3 is provided, in which the scale 20 is positioned underneath the condenser 17, in order to be able to weigh the condenser 17 and the water it contains. On the basis of an increase of the weight of the condenser 17 during the frying process, the controlling means are able to determine the quantity of water which is released. However, in order to find the moment at which the water content of the food corresponds to the required final water content, the initial quantity of the food must be known. For this purpose, the modified third fryer 3 may for example comprise an additional scale 20, or input means by means of which the user of the modified third fryer 3 is able to indicate the weight of the inserted food. An advantage of using two scales 20 for determining both the weight of the food and the weight of the released water during the frying process is that the accuracy of the process of finding the moment on which the food is ready is increased.

It is possible that the frying process comprises more than one step, wherein each step has a specific temperature profile. In such a case, for every step, a final water content associated with a ready state of the food is stored in the controlling means. It will be understood that an initial water content of one step corresponds to a final water level of a preceding step. Further, for every step, a reference value of the weight as detected by the scale 20 is determined, in the manner as described in the foregoing, wherein the values of the initial water content, the final water content, the initial weight of the food and the weight of the oil and the fryer components above the scale 20 are used. During the frying process, an actual value of the weight as detected by the scale 20 is checked and compared to the reference value. One step of the preparation process of the food is finished as soon as the actual value of the weight as detected by the scale 20 corresponds to the reference value, or is lower than the reference value.

The controlling means may be programmed such as to perform various checks on properties which may influence the operation of the fryer 1, 2, 3 and/or the frying process. For example, the value of the weight of the oil in the oil bowl 15 may be checked. In case the controlling means find that the weight of the oil is below a predetermined minimum or above a predetermined maximum, the controlling means transmit a signal in order to activate a warning device (not shown) for warning a user of the fryer 1, 2, 3, for example by producing noise. As long as the weight of the oil is below the predetermined minimum or above the predetermined maximum, the controlling means will prohibit activation of the heating elements 16 of the fryer 1, 2, 3.

Another important property to be checked during the frying process is the temperature. In the controlling means, a predetermined temperature profile is stored. In case of the frying process comprising more than one step, various predetermined temperature profiles are stored in the controlling means, wherein each predetermined temperature profile is associated with a particular step. At the start of the frying process, or at the start of a step of the frying process, checking of the temperature plays a role in heating the fryer 1, 2, 3 to a predetermined temperature. Further, during the frying process, the safety may be guarded by checking whether the temperature remains below a predetermined maximum.

As already mentioned in the foregoing, the fryer 1, 2, 3 may have a button which may be operated by a user in order to indicate a desired ready state of the food, and a button which may be operated by a user in order to indicate a desired crispness and/or brown level of the food. The crispness and/or brown level of the food are mainly determined by the temperature profile of the frying process. Therefore, according to the present invention, it is possible to obtain food having reached the ready state and at the same time having obtained a best possible crispness and/or brown level. There is hardly any need to find a compromise between these two features of prepared food, as the temperature of the frying process may be set on the basis of the desired crispness and/or brown level of the food, and the duration of the frying process may be determined on the basis of a difference between an initial water content and a final water content of the food, wherein the final water content is associated with the ready state of the food.

The input means of the fryer 1, 2, 3 do not necessarily need to comprise rotatable buttons. On the contrary, there are many manners in which information regarding the type of inserted food and information regarding user's desires may be transmitted to the controlling means. For example, a remote control may be used, wherein the fryer 1, 2, 3 may be provided with a recess for accommodating the remote control. A wireless input keyboard may be provided, which may also be used at a distance from the fryer 1, 2, 3. An important advantage of such an input keyboard is that the keys will not easily become greasy and/or dirty.

At least a portion of the controlling means and/or the input means may be removable from the fryer 1, 2, 3, so that it is easy to replace these components of the fryer 1, 2, 3. If that is the case, it is possible to easily update or adjust the information stored in the controlling means. The removable portion may for example comprise a chip card. The information stored in the controlling means may for example be adjusted to preferences of a certain country or continent in which the fryer 1, 2, 3 is used. According to another option, the information stored in the controlling means may be updated or adjusted by means of the Internet.

Preferably, the fryer 1, 2, 3 is provided with basket detecting means (not shown) for detecting whether the basket 14 has been placed in the oil bowl 15 or not, as it is not required to use the basket 14 for placing food in the fryer 1, 2, 3. The basket detecting means are activated before the scale 20 performs a measurement. In case the basket detecting means transmit a signal to the controlling means indicating that the basket 14 is present, the weight of the basket 14 is taken into account in processing the result of the measurement as performed by the scale 20. For this purpose, the weight of the basket 14 is stored in the controlling means. Contrariwise, in case the basket detecting means transmit a signal to the controlling means indicating that the basket 14 is absent, the weight of the basket 14 is not taken into account in processing the result of the measurement as performed by the scale 20.

It will be understood that in case additional food would be inserted in the fryer 1, 2, 3 during a frying process, a new situation would be created, in which the determined reference value of the weight as detected by the scale 20, which has been determined by the controlling means at the start of the frying process, is no longer relevant. Therefore, insertion of food during the frying process should be prohibited. Preferably, the fryer 1, 2, 3 comprises cover retaining means (not shown) for retaining the cover 13 of the fryer 1, 2, 3 during a frying process. Normally, after having been activated at the start of a frying process, the retaining means are not deactivated before the frying process is stopped by the controlling means, unless a manual interruption is performed by a user of the fryer 1, 2, 3. In case the cover 13 of the fryer 1, 2, 3 has been opened during the frying process, the determined parameters of the frying process may only remain unchanged if the quantity of food in the fryer 1, 2, 3 has not been altered.

An important advantage of the fryer 1, 2, 3 over a conventional fryer is that the fryer 1, 2, 3 is able to automatically stop the frying process, at the exact moment when the food is ready.

An important advantage of the method according to the present invention is that the moment in the frying process at which the food has reached a ready state is accurately determined. With the help of the results of measurements as performed by the scale 20 and information which is stored in the controlling means, it is possible to determine the moment at which the food is ready. All that the user of the fryer 1, 2, 3 needs to do is to indicate the type of the food which is inserted. It is not very likely that the user makes a mistake in this respect. When the method according to the present invention is applied, it is not necessary to collect additional information, for example information regarding for example the initial temperature of the food, because this property does not play a role in the process of determining the moment when the food is ready.

In an advantageous embodiment, the fryer 1, 2, 3 is provided with automatic lifting means (not shown) for lifting the basket 14 on receipt of a signal originating from the controlling means. In this embodiment, the controlling means are programmed so as to lift the basket 14 when the end of the frying process has been reached. In this way, it is prohibited that the food gets too well done, which may occur when the food remains in the oil. Further, in the embodiment of the fryer 1, 2, 3 comprising automatic lifting means, it is possible to lift the basket 14 between two steps of the frying process. For example, in case of the food comprising spring rolls, it is desirable to take the spring rolls out of the oil between two steps of the frying procedure, in order to allow for a process in which the jacket of the spring rolls is moistened. Hence, as a result of the presence of the automatic lifting means, the frying process can be performed in an optimal manner, wherein the basket 14 is lifted from the oil bowl 15 when necessary.

If automatic lifting means are present, the controller may be programmed to check the speed of the evaporation of water from the food during the frying process, and to check whether there is enough time to obtain the desired crispness and/or brown level. If it turns out that the speed of the evaporation of water is too high, it may be necessary to adjust the temperature profile in order to still obtain the desired crispness and/or brown level. In the process, the basket 14 may be lifted from the oil bowl 15 while the temperature of the oil is adjusted. After the oil temperature has been adjusted, the basket 14 may be lowered again.

In the foregoing, a fryer 1 for subjecting food to a frying process is disclosed. The fryer 1 comprises a housing 10, a basket 14 for receiving and containing food, and an oil bowl 15, which is capable of receiving the basket 14, and which may be filled with oil.

Furthermore, the fryer 1 comprises a scale 20 for performing weighing processes, which is positioned underneath the housing 10, and which is connected to controlling means for registering and processing results of the weighing processes as performed by the scale 20.

The controlling means are programmed such as to determine a moment when food is ready on the basis of input regarding the type of inserted food, an initial weight of the food and a required weight reduction of the food, which is associated with a required water content reduction. During a frying process, an actual weight of the food is determined in order to check whether the required weight reduction has been realized.

It will be clear to a person skilled in the art that the scope of the present invention is not limited to the examples discussed in the foregoing, but that several amendments and modifications thereof are possible without deviating from the scope of the present invention as defined in the attached claims.

The present invention may be applied in the context of a frying process, as described in the foregoing, but may also be applied in a baking process, for example baking of bread, or any other comparable preparation process. Further, it is possible to use the method according to the invention for the purpose of microwave heating, in which normally no preparation medium is applied, or microwave cooking, in which normally only a little water is applied.

In the foregoing, a fryer 1, 2, 3 has been described, in which the method according to the present invention for determining a moment in the preparation process when the food has reached the ready state is applied. The method according to the present invention may also be applied in other preparation devices, for example a microwave oven or a bread baking machine. An embodiment of such a microwave oven is diagrammatically shown in FIG. 5, in which the microwave oven is indicated by means of reference numeral 4.

The microwave oven 4 is for subjecting food to a microwave heating or cooking process. The microwave oven 4 comprises a housing 10 and a door (not shown) for closing the housing 10, wherein one side of the door is pivotably connected to the housing 10.

For the purpose of irradiating food with microwaves, the microwave oven 4 comprises an antenna 35 which is located inside the housing 10, at a top side of the microwave oven 4. At a bottom of the microwave oven 4, a dish 30 for supporting the food is provided. The dish 30 is rotatably arranged inside the housing 10, and is connected to a scale 20 by means of a mechanical connection, which is diagrammatically depicted in FIG. 5 and indicated by means of reference numeral 31. In this configuration, the scale 20 is capable of measuring the total weight of the dish 30, the mechanical connection 31 and the food, assuming that food has been placed on the dish 30. The food may be placed directly on the dish 30, but may also be placed on a plate, or may be located inside a container, wherein the container may further be filled with a little water, for example. If the latter is the case, the scale 20 measures the total weight of the dish 30, the mechanical connection 31, the container, the food and the water.

The scale 20 is connected to controlling means (not shown), which register and process the values of the weight as measured by the scale 20, and which may for example comprise a microcontroller. The controlling means are designed such as to use these values in a process of determining a moment in the microwave heating or cooking process when the food is ready. The way in which this process is performed, is comparable to the way as already described in the foregoing in the context of the fryer 1, 2, 3.

In the controlling means, the value of the total weight of the dish 30 and the mechanical connection 31 is stored. At the start of a microwave heating or cooking process, the scale 20 is activated to perform an initial measurement, wherein the scale 20 measures a total weight of the said components of the microwave oven 4 and the food and/or supporting plate or surrounding container and, if present, other fillings of the container which are placed on the dish 30. The result of the initial measurement is transmitted to the controlling means, which determine the value of the weight of the food and/or supporting plate or surrounding container and, if present, other fillings by calculating a difference between the stored value of the total weight of the dish 30 and the mechanical connection 31 and the measured value.

In order for the controlling means to be able to control the microwave heating or cooking process according to the desires of a user, the controlling means must be provided with information associated with these desires, as well as with information regarding the type of the food which is inserted. For this purpose, the microwave oven 4 is provided with input means (not shown), which are to be set by a user of the microwave oven 4. The input means may for example comprise two rotatable buttons, wherein the position of a first button is specific of the type of food which is inserted, and wherein the position of a second button is specific of a desired ready state of the food. The desired ready state may for example be indicated as medium, done, well done.

In the controlling means, information regarding an initial water content of the possible different types of food is stored. Furthermore, the controlling means contain information regarding a final water content of the possible different types of food, which final water content is associated with a ready state of the food. In order to be able to realize different ready states, different values of the final water content of the food are stored in the controlling means. As soon as a user has indicated the type of food which is inserted, the controlling means is able to find the associated initial water content of the food and the final water content of the food. On the basis of the value of the initial weight of the food, which has already been determined by the controlling means, and a difference between the initial water content and the final water content of the food, the controlling means are able to determine a required final weight of the food. The required final weight of the food is defined as the weight of the food at which the water content of the food corresponds to the final water content, in other words, the weight of the food at which the food is considered to be ready.

As already mentioned in the foregoing, the food may be surrounded by a container, which may contain a little water. The controlling means may be programmed to account for the influence the evaporation of the water has on the weight reduction as detected by means of the scale 20 during a microwave heating or cooking process. Further, the controlling means may be programmed to account for the weight of the container. Detecting means (not shown) may be provided for detecting the presence of a container, but it is also possible that the presence of a container is associated with certain types of food. As the weight of the container and, if present, the water may be small relative the weight of the food, it is also possible to ignore the weight of the container and the water, and still reach the predetermined ready state of the food.

The controlling means are able to determine a reference value of the weight as detected by the scale 20, which is associated with the required final weight of the food. The reference value is simply found by adding the total weight of the dish 30 and the mechanical connection 31, and, if appropriate, the weight of the plate or container and, if present, the water, to the required final weight of the food.

An actual value of the weight as detected by the scale 20 is regularly checked and compared to the reference value throughout the microwave heating or cooking process. As soon as it appears that the actual value of the weight corresponds to the reference value, or that the actual value of the weight is below the reference value, the controlling means stop the microwave heating or cooking process. At that point, the water content of the food has reached the level of the final water content which is associated with the ready state of the food, which means that the food has actually reached the ready state.

Microwave ovens having a scale 20 for determining the weight of inserted food are known. However, the controlling means of these known microwave ovens are programmed in another way than the controlling means of the microwave oven 4 according to the present invention. According to the state of the art, the controlling means are programmed so as to determine a preparation time on the basis of the weight of inserted food. Therefore, the weight of the food is only determined at the start of the microwave heating or cooking process. The controlling means are not programmed so as to actively check the state of the food in order to determine whether a ready state has been reached.

1. Method for determining a moment in a preparation process of food at which the food has reached a ready state, the method comprising the following steps:
determining a initial water content of the food;
determining a final water content of the food, which is associated with the ready state of the food;
determining an actual quantity of the food;
determining a required quantity of released water, i.e. a quantity of water to be released during the preparation process in order for the food to reach the ready state, on the basis of the determined actual quantity of the food and a difference between the initial water content of the food and the final water content of the food; and
performing measurements during the preparation process in order to determine a moment on which an actual quantity of released water corresponds to the required quantity of released water.

2. Method according to claim 1, comprising the following steps:
collecting the water which is released during the preparation process;
determining an actual quantity of released water; and
comparing the actual quantity of released water to the required quantity of released water in order to determine a moment in the preparation process when the actual quantity of released water corresponds to the required quantity of released water.

3. Method according to claim 1, comprising the following steps:
determining the extent to which the weight of the food needs to be reduced in order for the food to reach the ready state, on the basis of the difference between the initial water content of the food and the final water content of the food;
determining an initial weight of the food at the start of the preparation process;
determining a required final weight of the food on the basis of the determined initial weight of the food and the determined reduction of the weight of the food;
determining an actual weight of the food during the preparation process; and
comparing the actual weight of the food to the required final weight of the food in order to determine a moment in the preparation process when the actual weight of the food corresponds to the required final weight of the food.

4. Method according to claim 3, wherein the preparation process of the food takes place in a preparation device (1, 2, 3), the method comprising the following steps:
determining an empty weight of the preparation device (1, 2, 3), i.e. a weight of the preparation device (1, 2, 3) without inserted food;
inserting the food into the preparation device (1, 2, 3);
determining a total weight of the preparation device (1, 2, 3) and the inserted food; and
determining a weight of the inserted food by subtracting the empty weight of the preparation device (1, 2, 3) from the total weight of the preparation device (1, 2, 3) and the inserted food.

5. Method according to claim 3, wherein the preparation process of the food takes place in a preparation device (1, 2, 3) containing a preparation medium, the method comprising the following steps:
determining a total weight of the preparation device (1, 2, 3) and the preparation medium;
inserting the food into the preparation device (1, 2, 3);
determining a total weight of the preparation device (1, 2, 3), the preparation medium and the inserted food; and
determining a weight of the inserted food by subtracting the total weight of the preparation device (1, 2, 3) and the preparation medium from the total weight of the preparation device (1, 2, 3), the preparation medium and the inserted food.

6. Method according to claim 5, comprising the following steps:
determining a minimum weight of the preparation medium, which is associated with a minimum quantity of the preparation medium that needs to be present in the preparation device (1, 2, 3) in order for the preparation process to take place in a proper and/or safe manner;
determining an empty weight of the preparation device (1, 2, 3), i.e. a weight of the preparation device (1, 2, 3) without preparation medium and inserted food;
determining an actual weight of the preparation medium by subtracting the empty weight of the preparation device (1, 2, 3) from the total weight of the preparation device (1, 2, 3) and the preparation medium; and
comparing the actual weight of the preparation medium to the minimum weight of the preparation medium.

7. Method according to claim 5, comprising the following steps:
determining a maximum weight of the preparation medium, which is associated with a maximum quantity of the preparation medium that is allowed in the preparation device (1, 2, 3) in order for the preparation process to take place in a proper and/or safe manner;
determining an empty weight of the preparation device (1, 2, 3), i.e. a weight of the preparation device (1, 2, 3) without preparation medium and inserted food;
determining an actual weight of the preparation medium by subtracting the empty weight of the preparation device (1, 2, 3) from the total weight of the preparation device (1, 2, 3) and the preparation medium; and
comparing the actual weight of the preparation medium to the maximum weight of the preparation medium.

8. Method according to claim 1, further comprising the following steps:
defining a relation between a temperature profile of the preparation process and a crispness and/or brown level of the food; and
determining a temperature profile which needs to be realized during the preparation process in order for the food to obtain a predetermined crispness and/or brown level, on the basis of the defined relation between the temperature profile of the preparation process and the value of crispness and/or brown level of the food.

9. Method according to claim 8, comprising the following steps:
determining a speed at which the water is released from the food;
estimating an actual duration of the preparation process on the basis of the determined speed and the difference between the initial water content of the food and the final water content of the food;
comparing the estimated actual duration of the preparation process to a duration of the preparation process that is required on the basis of the temperature profile; and, in case a difference between the estimated actual duration of the preparation process and the required duration of the preparation process is found,
adjusting the temperature profile such that it is possible for the food to substantially simultaneously reach the ready state and the predetermined crispness and/or brown level.

10. Method according to claim 1, wherein the food is subjected to a frying process, during which the food is fried in a preparation medium such as oil, and which comprises at least two steps having specific temperature profiles.

11. Method according to claim 10, wherein the food is taken out of the preparation medium between two successive steps.

12. Preparation device (1, 2, 3) for subjecting food to a preparation process, comprising:
a housing (10) having a compartment (15) for receiving and containing food;
weighing means (20) for performing weighing processes in which at least a portion of the housing (10) having the compartment (15) is weighed; and
controlling means for registering and processing results of the weighing processes as performed by the weighing means (20), wherein the controlling means are programmed such as to perform the method according to claim 1.

13. Preparation device (1, 2, 3) according to claim 12, further comprising input means which are to be set by a user of the preparation device (1, 2, 3), wherein a manner in which the input means are set is specific of the type of food which is inserted in the preparation device (1, 2, 3).

14. Preparation device (1, 2, 3) according to claim 13, wherein a manner in which the input means are set is specific of a desired crispness and/or brown level of the food.

15. Preparation device (1, 2, 3) according to claim 13, wherein a manner in which the input means are set is specific of a desired ready state of the food.

16. Preparation device (1, 2, 3) according to claim 12, further comprising a condenser (17) for collecting water which is released during a preparation process of food.

17. Preparation device (1, 2, 3) according to claim 16, further comprising additional weighing means (20) which are positioned underneath the condenser (17).

18. Preparation device (1, 2, 3) according to claim 12, wherein the housing (10) comprises an inner housing (11) and an outer housing (12), wherein the inner housing (11) is located inside the outer housing (12), and wherein the weighing means (20) are located outside the inner housing (11), but inside the outer housing (12).

19. Preparation device (1, 2, 3) according to claim 12, further comprising retaining means for keeping the compartment (15) closed during the preparation process.

20. Preparation device (1, 2, 3) according to claim 12, being a fryer (1, 2, 3) which comprises the following components:
a basket (14) for receiving and containing food; and
an oil bowl (15) for receiving and containing a preparation medium such as oil, and for receiving and containing the basket (14).

21. Preparation device (1, 2, 3) according to claim 20, further comprising basket detecting means for detecting whether the basket (14) has been placed in the oil bowl (15) or not.

22. Preparation device (1, 2, 3) according to claim 20, further comprising automatic lifting means for lifting the basket (14) on receipt of a signal originating from the controlling means, wherein the signal is at least transmitted at an end of the preparation process.

23. Preparation device (4) for subjecting food to a preparation process, in particular a microwave oven (4), comprising:
a dish (30) for supporting food;
a scale (20) for performing weighing processes in which at least the dish (30) is weighed; and
controlling means for registering and processing results of the weighing processes as performed by the scale (20), wherein the controlling means are programmed such as to perform the method according to claim 1.

24. Preparation device (4) according to claim 23, further comprising input means which are to be set by a user of the preparation device (4), wherein a manner in which the input means are set is specific of the type of food which is inserted in the preparation device (4).

25. Preparation device (4) according to claim 24, wherein a manner in which the input means are set is specific of a desired ready state of the food.
